SEQUENCE LISTING (29 sequences)

SEQ ID NO: 1 - protein, 806 aa, Homo sapiens
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 2 - DNA, 3198 bp, Homo sapiens
ctgccactgccacctcgcggatcaggagccagcgttgttcgcccgacgcctcgctgccggtgggaggaagcgagagggaagccgcttgggctcttgtcgccgctgctcgcccaccgcctggaagagccgagccccggccagtcggtcgcttgccaccgctcgtagccgttacccgcgggccgccacagccgccggcgggagaggcgcgcgccatggcttctggagccgattcaaaaggtgatgacctatcaacagccattctcaaacagaagaaccgtcccaatcggttaattgttgatgaagccatcaatgaggacaacagtgtggtgtccttgtcccagcccaagatggatgaattgcagttgttccgaggtgacacagtgttgctgaaaggaaagaagagacgagaagctgtttgcatcgtcctttctgatgatacttgttctgatgagaagattcggatgaatagagttgttcggaataaccttcgtgtacgcctaggggatgtcatcagcatccagccatgccctgatgtgaagtacggcaaacgtatccatgtgctgcccattgatgacacagtggaaggcattactggtaatctcttcgaggtataccttaagccgtacttcctggaagcgtatcgacccatccggaaaggagacatttttcttgtccgtggtgggatgcgtgctgtggagttcaaagtggtggaaacagatcctagcccttattgcattgttgctccagacacagtgatccactgcgaaggggagcctatcaaacgagaggatgaggaagagtccttgaatgaagtagggtatgatgacattggtggctgcaggaagcagctagctcagatcaaagagatggtggaactgcccctgagacatcctgccctctttaaggcaattggtgtgaagcctcctagaggaatcctgctttacggacctcctggaacaggaaagaccctgattgctcgagctgtagcaaatgagactggagccttcttcttcttgatcaatggtcctgagatcatgagcaaattggctggtgagtctgagagcaaccttcgtaaagcctttgaggaggctgagaagaatgctcctgccatcatcttcattgatgagctagatgccatcgctcccaaaagagagaaaactcatggcgaggtggagcggcgcattgtatcacagttgttgaccctcatggatggcctaaagcagagggcacatgtgattgttatggcagcaaccaacagacccaacagcattgacccagctctacggcgatttggtcgctttgacagggaggtagatattggaattcctgatgctacaggacgcttagagattcttcagatccataccaagaacatgaagctggcagatgatgtggacctggaacaggtagccaatgagactcacgggcatgtgggtgctgacttagcagccctgtgctcagaggctgctctgcaagccatccgcaagaagatggatctcattgacctagaggatgagaccattgatgccgaggtcatgaactctctagcagttactatggatgacttccggtgggccttgagccagagtaacccatcagcactgcgggaaaccgtggtagaggtgccacaggtaacctgggaagacatcgggggcctagaggatgtcaaacgtgagctacaggagctggtccagtatcctgtggagcacccagacaaattcctgaagtttggcatgacaccttccaagggagttctgttctatggacctcctggctgtgggaaaactttgttggccaaagccattgctaatgaatgccaggccaacttcatctccatcaagggtcctgagctgctcaccatgtggtttggggagtctgaggccaatgtcagagaaatctttgacaaggcccgccaagctgccccctgtgtgctattctttgatgagctggattcgattgccaaggctcgtggaggtaacattggagatggtggtggggctgctgaccgagtcatcaaccagatcctgacagaaatggatggcatgtccacaaaaaaaaatgtgttcatcattggcgctaccaaccggcctgacatcattgatcctgccatcctcagacctggccgtcttgatcagctcatctacatcccacttcctgatgagaagtcccgtgttgccatcctcaaggctaacctgcgcaagtccccagttgccaaggatgtggacttggagttcctggctaaaatgactaatggcttctctggagctgacctgacagagatttgccagcgtgcttgcaagctggccatccgtgaatccatcgagagtgagattaggcgagaacgagagaggcagacaaacccatcagccatggaggtagaagaggatgatccagtgcctgagatccgtcgagatcactttgaagaagccatgcgctttgcgcgccgttctgtcagtgacaatgacattcggaagtatgagatgtttgcccagacccttcagcagagtcggggctttggcagcttcagattcccttcagggaaccagggtggagctggccccagtcagggcagtggaggcggcacaggtggcagtgtatacacagaagacaatgatgatgacctgtatggctaagtggtggtggccagcgtgcagtgagctggcctgcctggaccttgttccctgggggtggggcgcttgcccaggagagggaccaggggtgcgcccacagcctgctccattctccagtctgaacagttcagctacagtctgactctggacagggggtttctgttgcaaaaatacaaaacaaaagcgataaaataaaagcgattttcatttggtaggcggagagtgaattaccaacagggaattgggccttgggcctatgccatttctgttgtagtttggggcagtgcaggggacctgtgtggggtgtgaaccaaggcactactgccacctgccacagtaaagcatctgcacttgactcaatgctgcccgagccctcccttccccctatccaacctgggtaggtgggtaggggccacagttgctggatgtttatatagagagtaggttgatttattttacatgcttttgagttaatgttggaaaactaatcacaagcagtttctaaaccaaaaaatgacatgttgtaaaaggacaataaacgttgggtcaaaatggaaaaaaaaaaaaa

SEQ ID NO: 3 - protein, 12 aa, Homo sapiens
VRLVRGKREKAI

SEQ ID NO: 4 - protein, 12 aa, Mus musculus
VRLVRGKREKAI

SEQ ID NO: 5 - protein, 12 aa, Sus scrofa
VHLVRGKREKAI

SEQ ID NO: 6 - protein, 12 aa, Rattus norvegicus
VRLVRGKREKAI

SEQ ID NO: 7 - protein, 12 aa, Xenopus laevis
VRLVRGKREKAI

SEQ ID NO: 8 - protein, 12 aa, Danio rerio
VRLVRGKREKAI

SEQ ID NO: 9 - protein, 12 aa, Drosophila melanogaster
VHLVRAKREKAI

SEQ ID NO: 10 - protein, 12 aa, Caenorhabditis elegans
IRLVQAKREKAI

SEQ ID NO: 11 - protein, 12 aa, Caenorhabditis elegans
SRLVQAKREKAI

SEQ ID NO: 12 - protein, 12 aa, Arabidopsis thaliana
VRLVRGKREKSI

SEQ ID NO: 13 - protein, 12 aa, Arabidopsis thaliana
VRLVRGKREKSI

SEQ ID NO: 14 - protein, 12 aa, Arabidopsis thaliana
VRLVRGKREKSI

SEQ ID NO: 15 - protein, 12 aa, Capsicum annuum
VRLVRGKREKSI

SEQ ID NO: 16 - protein, 12 aa, Glycine max
VRLVRGKREKSI

SEQ ID NO: 17 - protein, 12 aa, Schizosaccharomyces pombe
VRLVRGNREKSI

SEQ ID NO: 18 - protein, 12 aa, Saccharomyces cerevisiae
IRLVRGNREKAI

SEQ ID NO: 19 - DNA, 20 bp, Homo sapiens
tttcttgtccgtggtgggat

SEQ ID NO: 20 - DNA, 20 bp, Homo sapiens
tttcttgtccatggtgggat

SEQ ID NO: 21 - DNA, 20 bp, Homo sapiens
tttcttgtccctggtgggat

SEQ ID NO: 22 - DNA, 20 bp, Homo sapiens
tttcttgtccgtggtgggat

SEQ ID NO: 23 - DNA, 20 bp, Homo sapiens
tttcttgtctgtggtgggat

SEQ ID NO: 24 - DNA, 20 bp, Homo sapiens
ctctttaaggcaattggtgt

SEQ ID NO: 25 - DNA, 20 bp, Homo sapiens
ctctttaaggaaattggtgt

SEQ ID NO: 26 - DNA, 20 bp, Homo sapiens
ccttcgtgtacgcctagggg

SEQ ID NO: 27 - DNA, 20 bp, Homo sapiens
ccttcgtgtaggcctagggg

SEQ ID NO: 28 - DNA, 15 bp, Homo sapiens
cctatcaaacgagag

SEQ ID NO: 29 - DNA, 20 bp, Homo sapiens
cctatcaaacaagaggtgag

(19) United States
(12) Patent Application Publication
(10) Pub. No.: US 2006/0014168 A1
(43) Pub. Date: Jan. 19, 2006

(54) Method of diagnosis of inclusion body myopathy-paget bone disease-frontotemporal dementia syndrome

(75) Inventors: Virginia E. Kimonis, Newton, MA (US); Giles D.J. Watts, Boston, MA (US)

Correspondence Address: DAVID S. RESNICK, 100 SUMMER STREET, NIXON PEABODY LLP, BOSTON, MA, 02110-2131, US

(73) Assignee: Children's Medical Center Corporation, Boston, MA (US)

(21) Appl. No.: 11/079,900

(22) Filed: Mar. 14, 2005

Related U.S. Application Data
(60) Provisional application No. 60/552,562, filed on Mar. 12, 2004.

(51) Int. Cl. C12Q 1/68

(52) U.S. Cl. 435/6

(57) ABSTRACT

The present invention is based on the discovery of a genetic basis for inclusion body myopathy-Paget bone disease-frontotemporal dementia syndrome (IBMPFD). We have determined that genetic alterations in the gene encoding vasolin containing protein (VCP) is responsible for IBMPDF syndrome. In particular, we have identified six missense mutations within VCP that are found in affected individuals. Accordingly, the present invention, provides nucleic acids encoding these mutations as well as methods for diagnosis of IBMPFD.

### CROSS-REFERENCE TO RELATED APPLICATIONS

This Application claims the benefit under 35 U.S.C §119(e) of U.S. Provisional Application No. 60/552,562, filed Mar. 12, 2004.

### GOVERNMENT SUPPORT

This invention was made with Government Support under Contract No. NIAMS R03 AR 46869, NINDS K02 NS02157 awarded by the National Institutes of Health. The Government has certain rights in the invention.

### FIELD OF THE INVENTION

The present invention is directed to diagnosis of dementia, muscle disease, Paget disease of bone, inclusion body myopathy and inclusion body myopathy—Paget bone disease—frontotemporal dementia syndrome (IBMPFD) by detecting mutations in the nucleic acid encoding Vasolin Containing Protein (VCP).

### BACKGROUND

Inclusion body myopathy (h-IBM) associated with Paget disease of bone (PDB) and frontotemporal dementia (FTD)—IBMPFD—is a rare, complex and ultimately lethal, autosomal dominant disorder (MIM 605382)¹. IBMPFD features adult-onset proximal and distal muscle weakness (clinically resembling limb girdle muscular dystrophy), early-onset PDB in most cases, and “premature” FTD². The disorder maps to chromosome 9p21-p12, but the genetic basis is unknown.

Vasolin containing protein (VCP), a member of the AAA-ATPase superfamily, has been associated with a wide variety of essential cellular protein pathways comprising nuclear envelope reconstruction, cell cycle, postmitotic Golgi reassembly, suppresssion of apoptosis and DNA damage response¹²⁻¹⁶. In addition to binding to expanded polyglutamine protein aggregates¹⁸, VCP has been postulated to play a pivotal role in ubiquitin-dependent protein degradation⁷.

Determination of a genetic basis for IBMPFD will allow for the development of diagnostics as well as means for predicting development of the syndrome.

### SUMMARY

The present invention is based on the discovery of a genetic basis for IBMPFD. It has been determined that one or more genetic alterations in the gene encoding vasolin containing protein (VCP) is responsible for IBMPDF syndrome. In particular, six missense mutations within VCP that are found in affected individuals have been identified. Accordingly, the present invention provides diagnostic methods to determine the presence or absence of IBMPFD in a patient. In addition, the methods of the invention can be used to predict the likelihood that an individual will develop IBMPFD.

In one embodiment, a method for diagnosing the presence or absence of IBMPFD in a patient is provided. The method, comprises obtaining a test biological sample from an individual, and analyzing a nucleic acid encoding vasolin containing protein (VCP) in the biological sample for alterations. An alteration in the nucleic acid encoding VCP compared to a control nucleic acid sample obtained from an individual not affected with the IBMPFD is indicative of the presence of IBMPFD.

The alterations to be detected by methods of the invention are nucleic acid mutations including missense and nonsense mutations as well as deletions, transpositions, insertions, and inversions that alter the structure, function, or expression of the VCP protein.

In methods of the invention, the control sample is sample containing a gene encoding VCP without an alteration (e.g. the VCP gene (AC004472) or mRNA coding sequence (SEQ ID NO: 2).

The biological sample obtained from a patient can be a bodily fluid sample such as blood, saliva, semen, vaginal secretion, cerebrospinal fluid and amniotic bodily fluid sample. Alternatively, or additionally the biological sample is a tissue sample such as epithelial, muscular, neuronal, bone, chorionic villous, or connective tissue sample. In another embodiment, the sample is a nucleic acid preparation obtained from human chromosome 9q13.

In one embodiment, a method for detecting the presence or absence of an alteration in a nucleic acid in a biological sample is provided. The method comprises a) analyzing a biological test sample containing a gene encoding VCP, b) comparing the results of the analysis of the biological test sample with the results of analysis of a control sample, and c) determining the presence or absence of the alteration in the test sample compared to the absence of the alteration in the control sample. The presence of the alteration in the test sample is indicative of the presence of IBMPFD.

In another embodiment, the method for diagnosing the presence or absence of IBMPFD in a patient comprises the steps of a) contacting a biological test sample obtained from the patient with a nucleic acid probe, where the nucleic acid probe detects at least one alteration in a gene encoding VCP, b) maintaining the biological test sample and the nucleic acid probe under conditions suitable for hybridization, c) detecting hybridization between the biological test sample and the nucleic acid probe; d) and comparing hybridization in the biological test sample from the patient to a control sample. The presence of hybridization between the biological test sample and the nucleic acid probe compared to the control sample is indicative of the presence of IBMPDF in the patient. Preferably the nucleic acid probe is labeled, with for example, a fluorescent, radioactive, or enzymatic label.

In one embodiment, the method for diagnosing the presence or absence of IBMPFD in a patient comprises the steps of a) performing a nucleic acid amplification of a biological test sample with oligonucleotide primers capable of amplifying a gene encoding VCP, b) analyzing the amplified nucleic acid fragments of the gene encoding VCP, and c) comparing the amplified nucleic acid fragments detected in step b) with amplified nucleic acid fragments of a control sample. The presence of an alteration in the biological test sample compared to the control sample is indicative of the presence of IBMPDF in the patient. In another embodiment, the amplified DNA fragments can be sequenced to detect the presence or absence of alterations.

In one embodiment, the alteration in the nucleic acid encoding VCP is an alteration that is present in Exon 3, Exon 5, or Exon 6 of a VCP gene (e.g., Genebank Accession AC004472). These Exons in the human VCP gene encode the CDC48 and L1 domains of the VCP protein (FIG. 3).

In one embodiment, the nucleic acid encoding VCP to be analyzed is the mRNA coding sequence of VCP (SEQ ID NO: 2). In one preferred embodiment, the alteration to be detected is a missense mutation such as 464 G>A; 464 G>C; 463 C>T; 695 C>A; 283 C>G; and/or 572 G>C (SEQ ID NO: 2).

Preferably, the alteration in the nucleic acid encoding VCP results in an amino acid change in VCP (SEQ ID NO: 1) that is selected from the group consisting of R155H, R155P, R155C, A232E, R95G, and R191Q.

The invention further provides for isolated VCP nucleic acids that encode a mutant VCP having an amino acid change selected from the group consisting of R155H, R155P, R155C, A232E, R95G, and R191Q.

In another embodiment, a method of diagnosing inclusion body myopathy is provided that comprises immunohistochemical staining of muscle sections with anti-VCP antibody. Localization of VCP within inclusion bodies is indicative of inclusion body myopathy. In one embodiment the method further comprises immunohistochemical staining with anti-ubiquitin antibody.

The invention also encompasses methods for predicting whether a human is likely to be affected with IBMPFD using the same methods as described herein for diagnostic purposes. The presence of an alteration in a nucleic acid encoding VCP as compared to a control indicates that the patient is likely to develop IBMPFD.

The present invention also provides kits for diagnosing the presence or absence of IBMPFD in a patient. The kits contain one ore more reagents for detecting an alteration in a nucleic acid encoding VCP in a biological sample obtained from a patient. The kits, for example, contain nucleic acid probes or primers capable of detecting the alterations described herein. Kits that comprise anti-VCP antibodies are also provided, For example the kit comprises anti-VCP antibodies that specifically interact only with a mutant VCP, e.g. having an amino acid change or deletion, while not interacting with wild type VCP (SEQ ID NO: 1). In one preferred embodiment, such antibodies specifically interact with VCP having at least one of the following mutations selected from the group consisting of R155H, R155P, R155C, A232E, R95G, and R191Q.

The kits further comprise instructions for use.

### BRIEF DESCRIPTION OF THE FIGURES

FIG. 1 shows the pedigrees of thirteen families with Inclusion Body Myopathy Associated with Paget Disease of the Bone and Frontotemporal Dementia: labeled families 1-7, families 9-11, family 13, and families 15-16. Squares indicate male family members, and circles female family members. Arrows indicate probands, and symbols with a slash indicate deceased family members. [symbol] represents inclusion body myopathy, [symbol] represents Paget disease of the bone and [symbol] represents frontotemporal dementia. Only clinically diagnosed family members are shown without ages due to confidentiality issues.

FIGS. 2a to 2f show the staining of normal and diseased human muscle with polyclonal anti-VCP antibody: FIG. 2a shows Normal muscle. VCP is prominently located in small endomysial capillaries (right arrow). In muscle fibers VCP accumulates with lipofuscin granules (left arrow) at the periphery and more diffusely, at low levels, in the cytoplasm. FIG. 2b shows sporadic inclusion body myositis (s-IBM): VCP is present in material in a vacuole (arrow) and in small accumulations in the muscle fiber cytoplasm. FIG. 2c shows s-IBM: VCP is strongly stained in endomysial inflammatory cells surrounding a muscle fiber. FIG. 2d shows s-IBM: VCP is up regulated in regenerating muscle fibers. FIG. 2e shows IBMPFD: Large focal inclusion (arrow) within muscle fiber contains VCP. FIG. 2f shows IBMPFD: Multiple small foci are present within a muscle fiber. (Magnification ×540)

FIGS. 3a to 3b show the mutations in the valosin containing gene (VCP) in patients with Inclusion Body Myopathy Associated with Paget Disease of the Bone and Frontotemporal Dementia. FIG. 3a shows the functional domains and mutations of the VCP gene. Arrows indicate the places where mutants occur relative to the exon-intron structure, where the exons are numbered 1 to 17. The relative position of N-domain (CDC48), flexible linker (L1), first AAA ATPase domain (D1), linker region (L2), second AAA ATPase domain (D2) and C-domain (C-terminal) are indicated, the 5′ and 3′ UTRs are represented in white. FIG. 3b shows the species conservation of amino acid residues mutated in IBMPFD, where the highlighted region indicates identical residues.

FIGS. 4a to 4e show mutations in the valosin containing gene (VCP) in patients with Inclusion Body Myopathy Associated with Paget Disease of the Bone and Frontotemporal Dementia. Sequencing chromatograms of genomic DNA from patients are shown. Since IBMPFD is dominant, all chromatograms show two overlapping peaks at the same locus (arrow) denoting heterozygous mutations (FIG. 4a to 4e). None of these mutations were detected in >90 control DNA samples (180 alleles). Further, from the 13 IBMPFD families, 61 affected and 62 unaffected individuals, respectively, were tested for the VCP mutations. Mutations were found in all of the affected but none of the unaffected individuals and demonstrated 100% cosegregation with the disease phenotype.

FIG. 5 shows a table illustrating the haplotype analysis of the IBMPFD families identified two ancestral, disease-associated haplotypes, distinguishing families 1, 3, 7, and 16 (Group A) of English/American origin from families 2 and 5 (Group B) of German/English origin. The Group A haplotype was 14-13-12-9-10-1-4-13-15-18-17 for markers D9S1118, D9S304, D9S165, D9S1878, D9S1805, D9S163, D9S1804, D9S1791, D9S50, D9S1874 and D9S2148, respectively. The Group B haplotype contained the 5-10-17-10-4-4 core haplotype at markers D9S304, D9S165, D9S1878, D9S1805, D9S163 and D9S1804, respectively. The minimal shared haplotype (D9S304 to D9S1804) represented a physical distance of approximately 3.6 Mb. Note family 10 is too small for haplotype analysis to determine the disease haplotype, but does not share a haplotype with other families. Families are assigned an arbitrary number and therefore are not sequential. *Base numbers relative to ATG start.

FIG. 6 shows the amino acid sequence of VCP (SEQ ID NO: 1).

FIG. 7 shows the mRNA coding sequence of VCP, NM_{—}007126 (SEQ ID NO: 2)

### DESCRIPTION OF THE INVENTION

As described herein, it has been determined that genetic alterations in the gene encoding vasolin containing protein (VCP) are associated with IBMPDF syndrome.

The term “VCP”, as used herein, refers to vasolin containing protein and is also known as CDC48 or p97. VCP is a ubiquitous protein and is a member of the AAA-ATPase super family, wherein “AAA” refers to ATPase Associated with a variety of cellular Activities. VCP is characterized by the presences of two conserved energy generating ATPases. Structurally VCP is divided into several domains: a cofactor (CDC48) and poly ubiquitin binding N domain (aa 1-187), N-D1 linker, D1 weak ATPase (aa 209-460), flexible D1-D2 linker, D2 the major ATPase (aa 481-761) and C (aa 762-806) domains (SEQ ID NO: 1).

The phrase “nucleic acid encoding VCP gene” as referred to throughout the specification refers to genomic DNA and RNA sequences with or without introns, promoters, enhancers and other regulatory sequences that are related to VCP gene expression. The genomic sequence of VCP is found at gene bank accession number AC004472; GeneID: 7415, which maps to 9q13-p12 (Locus tag: HGNC: 12666; MIM: 601023). The mRNA sequence encoding VCP is found at gene bank accession NM_{—}007126.

The term “alteration” refers to mutations including missense, nonsense, deletions, insertions, inversions and transpositions which alter either the structure, function, or expression of the VCP protein. Preferably the “alteration” is not a loss-of-function mutation.

The biological sample used as a source material for isolating the nucleic acids in the instant invention include, but are not limited to, solid materials (e.g., tissue, cell pellets, biopsies, bone) and biological fluids (e.g. blood, saliva, amniotic fluid, mouth wash, urine).

The nucleic acid molecules of the invention include DNA and RNA and can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001. For example, nucleic acid molecules can be isolated from a biological sample containing VCP RNA using the techniques of cDNA cloning and subtractive hybridization. The nucleic acid molecule can also be isolated from a cDNA library using a homologous probe. In addition, nucleic acid molecules can be isolated from a biological sample containing genomic DNA or from a genomic library. Suitable biological samples include, but are not limited to, whole organisms, organs, tissues, blood and cells. The method of obtaining the biological sample will vary depending upon the nature of the sample.

One skilled in the art will realize that genomes can be subject to slight allelic variations between individuals. Therefore, the isolated nucleic acid molecule is also intended to include allelic variations, so long as the sequence is a functional derivative of the VCP coding sequence. When a VCP allele does not encode the identical sequence to that found in SEQ ID NO: 2, it can be isolated and identified as VCP using the same techniques used herein, and especially PCR techniques to amplify the appropriate gene with primers based on the sequences disclosed herein.

One skilled in the art will realize that organisms other than humans can contain VCP genes (for example, eukaryotes; more specifically, mammals, rodents, worms (preferably, C. elegans), insects (preferably, fruit flies, Drosophila) birds, fish, yeast, and plants; more specifically, gorillas, rhesus monkeys, and chimpanzees). The invention is intended to include, but not be limited to, VCP nucleic acid molecules isolated from the above-described organisms.

The diagnostic and screening methods of the present invention encompass detecting the presence, or absence of, an alteration in a VCP gene wherein the alteration in the gene results in IBMPDF in a patient. For example, the diagnostic and screening methods of the present invention are especially useful for diagnosing the presence or absence of IBMPDF in a patient suspected of being at risk for developing IBMPDF based on family history, or a patient in which it is desired to diagnose IBMPDF.

As used herein the term “patient” is intended to encompass mammals including, but not limited to, vertebrate animals, including monotremes, marsupials and placental, that suckle their young and either give birth to living young (eutherian or placental mammals) or are egg-laying (metatherian or nonplacental mammals). Examples of mammalian species include primates (e.g., humans, monkeys, chimpanzees, baboons), rodents (e.g., rats, mice, guinea pigs, hamsters) and ruminants (e.g., cows, horses).

The screening method of the invention allows a presymptomatic diagnosis, including prenatal diagnosis, of the presence of an alteration in a gene encoding VCP in individuals, and thus an opinion concerning the likelihood that such individual would develop IBMPDF. This is especially valuable for individuals with a family history of the syndrome. Early diagnosis is also desired to maximize appropriate timely intervention.

In the methods of screening, a bodily fluid (e.g., blood, saliva, amniotic fluid) or tissue (e.g., neuronal, chorionic villous) sample would be taken from the individual and screened for (1) the presence or absence of the “normal” VCP gene; (2) the presence or absence of “normal” VCP mRNA and/or (3) the presence or absence of wild type VCP protein.

To detect VCP protein in methods of the invention, anti-VCP antibodies can be produced by methods well known to those skilled in the art. The term “antibody” is intended to include whole antibodies, e.g., of any isotype (IgG, IgA, IgM, IgE, etc), and includes fragments thereof which are also specifically reactive with VCP protein or VCP mutant protein. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody” includes polyclonal, monoclonal, or other purified preparations of antibodies and recombinant antibodies. The term “antibody” is further intended to include humanized antibodies, bispecific antibodies, and chimeric molecules having at least one antigen binding determinant derived from an antibody molecule. In a preferred embodiment, the antibody is detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

Mutant VCP proteins can be detected for the purpose of diagnosing or predicting onset of IBMPDF using anti-VCP antibodies by any means known to those in the art, for example by immunohistochemistry, Western Blot analysis, or Elisa. Alternatively, mass spectrometric methods can be used to determine the presence or absence of a mutant VCP. Preferably the mutant VCP has one or more of the following amino acid changes as compared to SEQ ID NO: 1: R155H, R155P, R155C, A232E, R95G, or R191Q.

In methods of the invention, alterations in a nucleic acid encoding VCP can be detected from nucleic acids isolated from a biological test sample using techniques such as direct analysis of isolated nucleic acids such as Southern Blot Hybridization (DNA) or direct nucleic acid sequencing (Molecular Cloning: A Laboratory Manual, 3rd Ed., Sambrook and Russel, Cold Spring Harbor Laboratory Press, 2001). Test samples suitable for use in the present invention encompass any sample containing nucleic acids, either DNA or RNA. For example, a test sample of genomic DNA is obtained from a human suspected of having IBMPDF. The test sample can be from any source which contains genomic DNA, such as a bodily fluid or tissue sample.

In one embodiment, the biological test sample of DNA is obtained from bodily fluids such as blood, saliva, semen, vaginal secretions, cerebrospinal and amniotic bodily fluid samples. In another embodiment, the biological test sample of DNA is obtained from tissue such as chorionic villous, neuronal, epithelial, muscular, bone and connective tissue. DNA can be isolated from the test samples using standard, art-recognized protocols (see, for example, Breakefield, et al., J. Neurogenetics 3:159-175 (1986)). The DNA sample is examined to determine whether a alteration associated with IBMPDF is present or absent. The presence of the alteration is indicated by hybridization of a probe that specifically detects the alteration to a nucleic acid encoding VCP, such as the VCP gene in the genomic DNA. Specific hybridization can be detected under high stringency conditions. “High stringency conditions” and “moderate stringency conditions” for nucleic acid hybridizations are explained on pages 2.10.1-2.10.16 and pages 6.3.1-6 in Current Protocols in Molecular Biology (Current Protocols in Molecular Biology, Ausubel, F. et al., eds., John Wiley & Sons, 1995) the teachings of which are hereby incorporated by reference. The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high or moderate stringency conditions can be determined empirically.

Other hybridization methods such as Northern analysis or slot blot analysis (see Current Protocols in Molecular Biology, Ausubel, F. et al., eds., John Wiley & Sons, 1995) can also be used to diagnose IBMPDF. For Northern analysis or slot blot analysis, a sample of RNA is obtained from the patient. Specific hybridization of a nucleic acid probe that hybridizes to an alteration in the RNA from the individual is indicative of the presence or absence of the alteration that is associated with IBMPDF and is, therefore, diagnostic for the syndrome.

In one embodiment, the alteration to be detected by methods of the invention is a missense mutation such as 464 G>A; 464 G>C; 463 C>T; 695 C>A; 283 C>G; and/or 572 G>C using SEQ ID NO: 2.

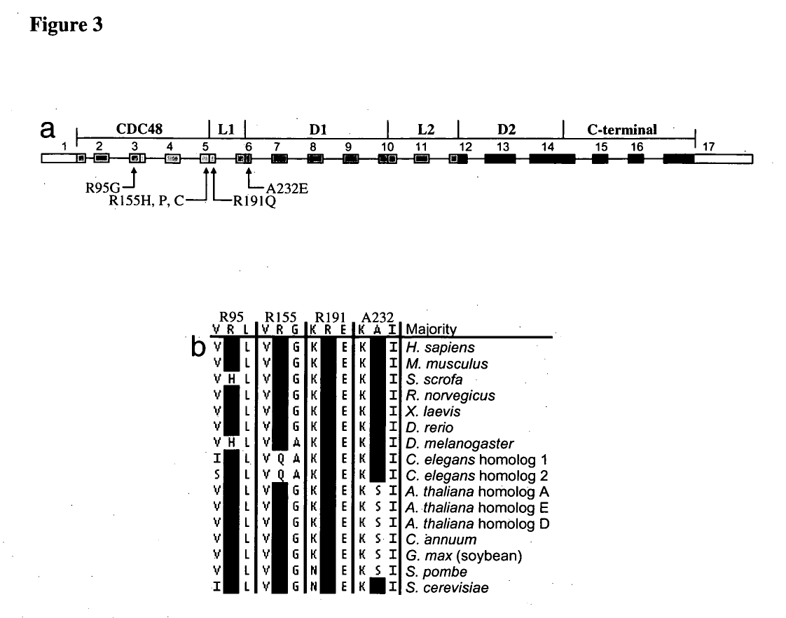

In another embodiment, the alteration in the nucleic acid encoding VCP is an alteration that is present in Exon 3, Exon 5, or Exon 6 of a VCP gene (e.g., Genebank Accession AC004472). These Exons in the human VCP gene encode the CDC48 and L1 domains of the VCP protein (FIG. 3).

In another embodiment of the invention, deletion analysis by restriction digestion can be used to detect a deletion in a VCP gene, if the deletion in the gene results in the creation or elimination of a restriction site. For example, a test sample containing genomic DNA is obtained from the patient. After digestion of the genomic DNA with an appropriate restriction enzyme, DNA fragments are separated using standard methods, and contacted with a probe specific for the VCP gene or cDNA. The digestion pattern of the DNA fragments indicates the presence or absence of the alteration associated with IBMPDF.

An alternative method useful according to the present invention for direct analysis of the VCP alterations is the INVADER® assay (Third Wave Technologies, Inc (Madison, Wis.). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535).

Preferably, a PCR based techniques are used. After PCR, the alterations in the VCP encoding nucleic acids can be identified using, for example direct sequencing with radioactively or fluorescently labeled primers; single-strand conformation polymorphism analysis (SSCP), denaturating gradient gel electrophoresis (DGGE); and chemical cleavage analysis, all of which are explained in detail, for example, in the Molecular Cloning: A. Laboratory Manual, 3rd Ed., Sambrook and Russel, Cold Spring Harbor Laboratory Press, 2001; also in U.S. Patent Application Publication 2004/0265849, which is herein incorporated by reference in its entirety. Generally the alteration is detected by comparing the test sample taken from the individual suspected to be affected with IBMPFD with a control sample, preferably several control samples taken from the individuals who are not affected with IBMPFD. The alteration can also be detected using published sequences as a gold standard bearing in mind that errors and non-disease-causing alterations are possibly present in the published sequence.

The VCP alterations are preferably analyzed using methods amenable for automation such as the different methods for primer extension analysis. Primer extension analysis can be preformed using any method known to one skilled in the art including PYROSEQUENCING™ (Uppsala, Sweden); Mass Spectrometry including MALDI-TOF, or Matrix Assisted Laser Desorption Ionization—Time of Flight; genomic nucleic acid arrays (Shalon et al., Genome Research 6(7):639-45, 1996; Bernard et al., Nucleic Acids Research 24(8):1435-42, 1996); solid-phase mini-sequencing technique (U.S. Pat. No. 6,013,431, Suomalainen et al. Mol. Biotechnol. June; 15(2):123-31, 2000); ion-pair high-performance liquid chromatography (Doris et al. J. Chromatogr. A May 8;806(l):47-60, 1998); and 5′ nuclease assay or real-time RT-PCR (Holland et al. Proc Natl Acad Sci USA 88: 7276-7280, 1991), or primer extension methods described in the U.S. Pat. No. 6,355,433. Nucleic acids sequencing, for example using any automated sequencing system and either labeled primers or labeled terminator dideoxynucleotides can also be used to detect the VCP alterations. Systems for automated sequence analysis include, for example, Hitachi FMBIO® and Hitachi FMBIO® II Fluorescent Scanners (Hitachi Genetic Systems, Alameda, Calif.); Spectrumedix® SCE 9610 Fully Automated 96-Capillary Electrophoresis Genetic. Analysis System (SpectruMedix LLC, State College, Pa.); ABI PRISM® 377 DNA Sequencer; ABI® 373 DNA Sequencer; ABI PRISM® 310 Genetic Analyzer; ABI PRISM® 3100 Genetic Analyzer; ABI PRISM® 3700 DNA Analyzer (Applied Biosystems, Headquarters, Foster City, Calif.); Molecular Dynamics FluorImager™ 575 and SI Fluorescent Scanners and Molecular Dynamics FluorImager™ 595 Fluorescent Scanners (Amersham Biosciences UK Limited, Little Chalfont, Buckinghamshire, England); GenomyxSC™ DNA Sequencing System (Genomyx Corporation (Foster City, Calif.); Pharmacia ALF™ DNA Sequencer and Pharmacia ALFexpress™ (Amersham Biosciences UK Limited, Little Chalfont, Buckinghamshire, England).

PCR, nucleic acid sequencing and primer extension reactions for one nucleic acid sample can be performed in the same or separate reactions using the primers designed to amplify and detect the VCP alterations.

A probe or primer typically is a substantially purified oligonucleotide or PNA oligomer. Such oligonucleotides typically comprises a region of complementary nucleotide sequence that hybridizes under stringent conditions to at least about 8, 10, 12, 16, 18, 20, 22, 25, 30, 40, 50, 60, 100 (or any other number in-between) or more consecutive nucleotides in a target nucleic acid molecule. Depending on the particular assay, the consecutive nucleotides can either include the target alteration position, or be a specific region in close enough proximity 5′ and/or 3′ to the alteration position to carry out the desired assay.

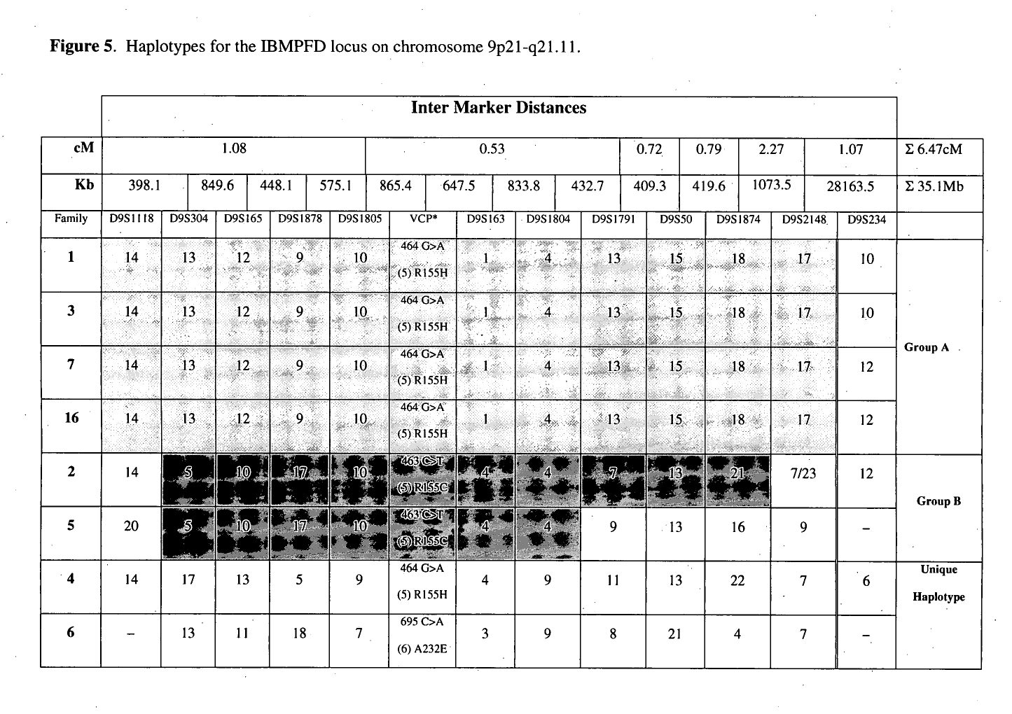

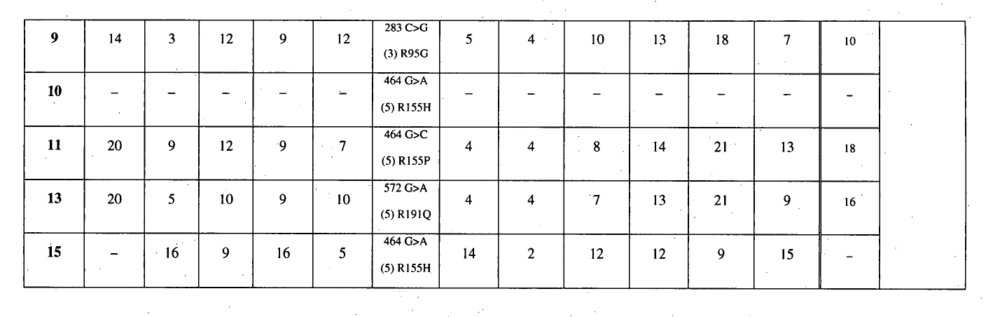

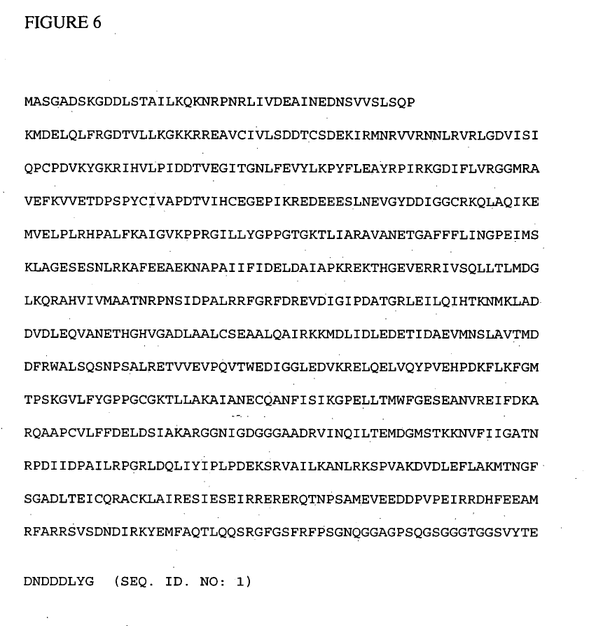

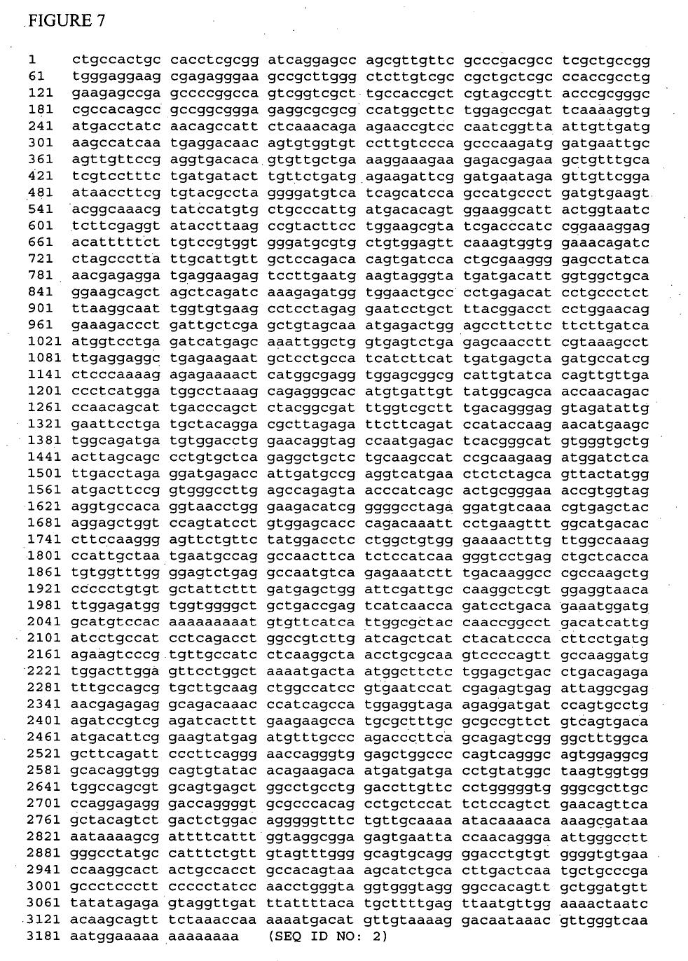

Other preferred primer and probe sequences can readily be determined using the transcript sequences, genomic sequences, and alteration context sequences (FIG. 5). It will be apparent to one of skill in the art that such primers and probes are directly useful as reagents for genotyping the alterations of the present invention, and can be incorporated into any kit/system format.

In order to produce a probe or primer specific for an alteration-containing sequence, the gene/transcript and/or context sequence surrounding the alteration of interest is typically examined using a computer algorithm which starts at the 5′ or at the 3′ end of the nucleotide sequence. Typical algorithms will then identify oligomers of defined length that are unique to the gene/alteration context sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length.

While the design of each primer or probe depends on variables such as the precise composition of the nucleotide sequences flanking an alteration position in a target nucleic acid molecule, and the length of the primer or probe, another factor in the use of primers and probes is the stringency of the condition under which the hybridization between the probe or primer and the target sequence is performed. Higher stringency conditions use buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions use buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an alteration specific probe are as follows: Prehybridization with a solution containing 5×standard saline phosphate EDTA (SSPE), 0.5% NaDodSO₄ (SDS) at 55° C., and incubating probe with target nucleic acid molecules in the same solution at the same temperature, followed by washing with a solution containing 2×SSPE, and 0.1% SDS at 55° C. or room temperature.

In one embodiment, the invention provides a nucleic acid chip including nucleic acids encoding normal and mutant VCP proteins for the screening of individual affected with IBMPFD. Such chip can include any number of other disease causing gene alterations. Methods and techniques applicable to array synthesis have been described in U.S. Ser. No. 09/536,841, WO 00/58516, U.S. Pat. Nos. 412,087, 6,147,205, 6,262,216, 6,310,189, 5,889,165, and 5,959,098, 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes. Additional methods of sample preparation and techniques for reducing the complexity of a nucleic sample are described, for example, in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays on the chips have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described, for example, in U.S. Pat. Nos. 5,871,928, 5,874,219, 6,045,996 and 6,386,749, 6,391,623 each of which are incorporated herein by reference

Examples of methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22:1859; and the solid support method described in U.S. Pat. No. 4,458,066.

In one embodiment, the invention further provides an isolated VCP nucleic acid encoding a mutant VCP having an amino acid change selected from the group consisting of R155H, R155P, R155C, A232E, R95G, and R191Q. The isolated nucleic acid can comprise, for example, a full-length gene or transcript, such as isolated from genomic DNA (e.g., by cloning or PCR amplification), a cDNA molecule, or an mRNA transcript molecule. Furthermore, fragments of such full length genes and transcripts that contain one or more alterations disclosed herein are also encompassed by the invention. As an example, the nucleic acid molecule may contain any one of the following nucleotide changes in the nucleic acid encoding VCP (NM_{—}007126) 464 G>A; 464 G>C; 463 C>T; 695 C>A; 283 C>G; and 572 G>C. Sequences complimentary to an isolated VCP nucleic acid encoding a mutant VCP having an amino acid change selected from the group consisting of R155H, R155P, R155C, A232E, R95G, and R191Q are also provided.

The present invention encompasses nucleic acid analogs, synthetic, or non-naturally occurring nucleotides or structural elements or other alternative/modified nucleic acid chemistries known in the art. Such nucleic acid analogs are useful, for example as probes or primers useful for detecting the alterations of the invention. The use of PNA oligomers are specifically contemplated, for example for use in array based technologies.

In another embodiment, the invention provides for a method for diagnosing inclusion body myopathy, such as sporadic inclusion body myositis (s-IBM) or IBMPFD, comprising immunohistochemical staining of muscle sections with anti-VCP antibody, wherein localization of VCP within inclusion bodies is indicative of inclusion body myopathy.

The invention further provides for kits comprising one or more primer pairs capable of amplifying the VCP nucleic acid; buffer and nucleotide mix for the PCR reaction; appropriate enzymes for PCR reaction in same or separate containers as well as an instruction manual defining the PCR conditions, for example, as described in this application, as well as listing the known IBMPFD causing VCP mutations to assist in the diagnosis. The kit may further comprise nucleic acid probes or additional primers for detecting specific VCP mutations as described in this application or identified from new patients, either in dry form in a tube or a vial or in a buffer. In one embodiment, the kit further comprises primers capable of amplifying disease related genes.

In one embodiment, the kit comprises arrays/microarrays of nucleic acid molecules or beads that contain one or more probes, primers, or other detection reagents for detecting one or more alterations of the invention.

The invention is further directed to mutant VCP encoding nucleic acids. In the preferred embodiment, these mutations are as shown in FIG. 5.

### EXAMPLES

### Example 1

Inclusion Body Myopathy Paget Bone Disease Frontotemporal Dementia Syndrome is Caused by Mutated Valosin Containing Protein.

Patients and Methods

Clinical Evaluations

Written consent from each subject was approved by the Springfield, Ill. Committee for Research Involving Human Subjects, and by Children's Hospital, Boston, Mass. Volunteers were all over age 18 years because IBMPFD manifests in adults. IBMPFD muscle phenotype is of variable severity, and mild asymmetry characterize the muscle weakness of IBMPFD^{1,2}. Myopathic features include variation in muscle fiber size, mildly increased endomysial connective tissue, and large focal regions of “myopathic grouping” commonly seen in h-IBM2. Rimmed vacuolar inclusions were noted in approximately 35% of muscle biopsy specimens analyzed in the 13 families. Electron microscopy (EM) from IBMPFD biopsies showed atrophic and vacuolated muscle fibers containing abundant nuclear and cytoplasmic, paired helical filaments (PHF) with congophilia, accumulations of phosphorylated tau, apolipoprotein E (ApoE), and excessive β-amyloid precursor protein epitopes^{1,2}.

Similarly, EM of PDB osteoclasts in 4 affected individuals from IBMPFD family 11 identified seemingly characteristic nuclear and cytoplasmic PHF inclusions²⁹

Chromosomal Mapping

Peripheral blood DNA was extracted using the PureGene DNA isolation kit (Gentra Systems, Inc., Minneapolis, Minn.). IBMPFD linkage to chromosome 9p21.1-p12 was known in 4 families,² and confirmed in the 9 new kindreds. The disease haplotype was constructed for each family to identify the critical locus.²

Assessments of Candidate Genes

A candidate approach involved genes prioritized and selected by their expression patterns and putative functions. Sequences provided by the Human Genome Project were identified containing Genethon markers that mapped to the disease region. Each sequence was then assessed using the National Center for Biotechnology Information BLAST search program (http://www.ncbi.nlm.nih.gov/BLAST) to determine the exon/intron structures of candidate genes—including the gene encoding the Valosin Containing Protein (VCP) (MIM #601023).

Mutation Analysis of the VCP Gene

Mutation analysis of the VCP gene (NM_{—}007126) initially involved two affected individuals from each of the 13 families. Non-affected individuals and unrelated relatives served as controls. PCR primers for genomic DNA were designed to include at least 50 bp of intron sequence, from either side of the exon, for all 17 exons. Sequences longer than 1000 bp were divided into multiple, overlapping segments for amplification. PCR products were gel purified using the Gel Extraction Kit (Qiagen, Valencia, Calif.), and sequenced with an ABI 377 sequencer, using a dRhodamine terminator cycle sequencing kit (Applied BioSystems Inc., Foster City, Calif.). Sequence and trace file comparisons were carried out using Lasergene 99 software (DNAStar Inc., Madison, Wis.). We then screened >180 additional control chromosomes by denaturing high-performance liquid chromatography (dHPLC) (Transgenomic, Inc. Omaha, Nebr.) and by restriction digests (see below) for any base changes in exons 3, 5 and 6, where mutations were identified, to rule out the possibility that the base changes were common polymorphisms. No base changes were detected in these control chromosomes.

Cosegregation Studies

Restriction site mapping from PCR amplified exons was utilized to confirm that the mutation cosegregated with disease in the families containing mutation 695 C>A (family 6) which destroys an MfeI site, 283 C>G (family 9) destroys an RSAI restriction site and 464 C>T (family 11) which creates a BSSKI restriction site. Cosegregation for mutations 463 C>T, 464 G>A, and 572 G>C was confirmed using dHPLC in a blinded study of 79 individuals from 7 families.

Immunohistochemistry

To determine the presence of VCP in postmortem sections from normal, IBM and IBMPFD muscle sections were subjected to immunohistochemistry with anti-VCP polyclonal antibody. Immunohistochemistry was performed as described previously². The immune reactivity was detected by light microscopy using horseradish peroxidase.

Results

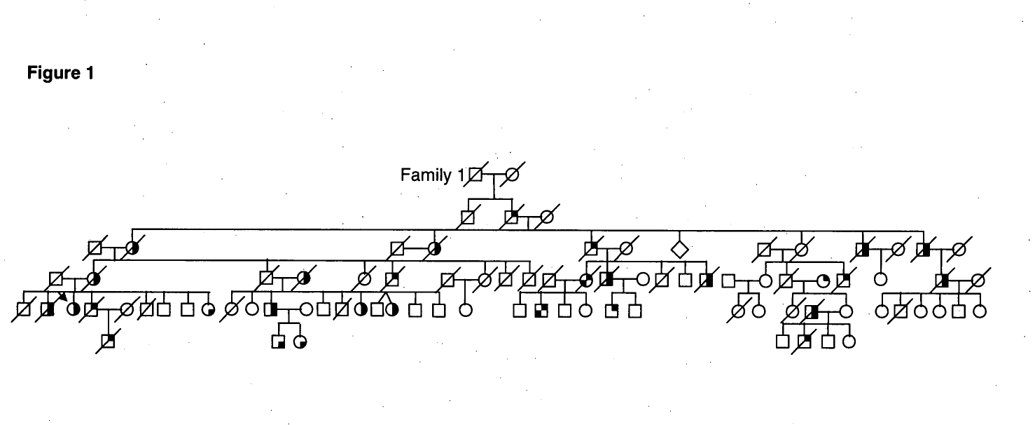

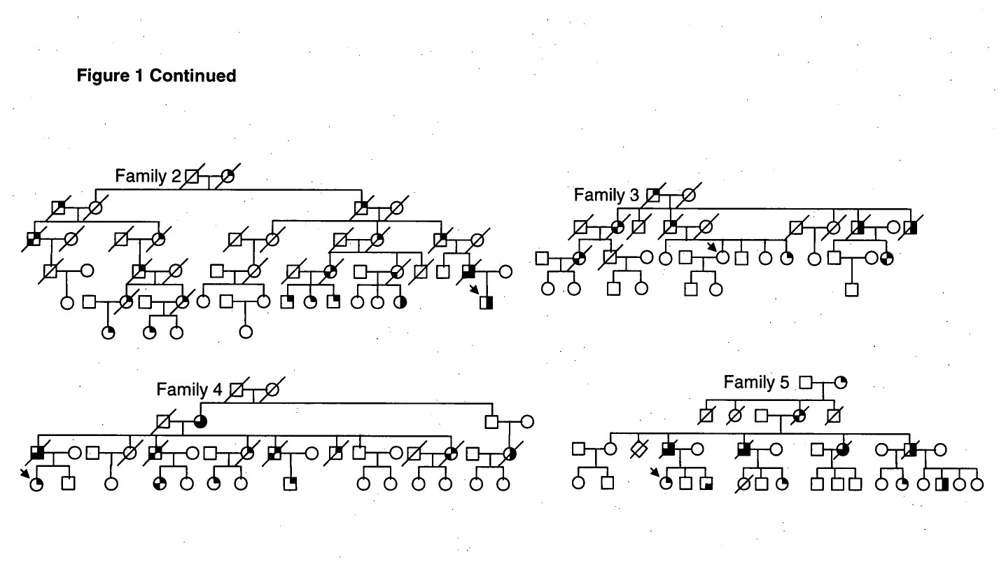

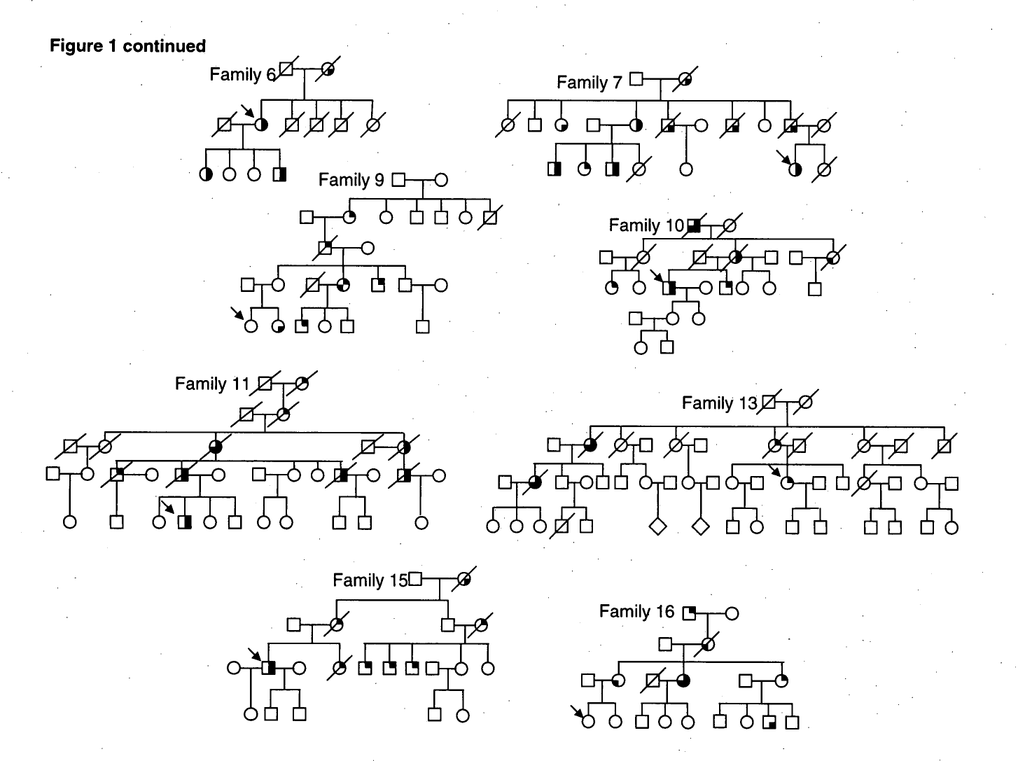

Within 13 families (12 from the United States and 1 from Canada, FIG. 1), 82% of patients had myopathy, 49% had PDB, and 30% had early-onset FTD. The mean age of presentation was 42 years for both IBM and PDB, whereas FTD typically presented at age 53 years. In IBMPFD myopathic muscle and Pagetic osteoclasts, inclusions appear similar—suggesting disruption within the same pathological pathway.

Haplotype analysis of the IBMPFD families identified two ancestral, disease-associated haplotypes, distinguishing families 1, 3, 7, and 16 (Group A) of English/American origin from families 2 and 5 (Group B) of German/English origin (FIG. 5). The predominant IBMPFD haplotype of Group A includes a core haplotype flanked by D9S1118 and D9S234 (6.47 cM, 35.1 Mb), probably transmitted from a shared ancestor (“founder”). However, it is unknown whether this individual emigrated to the United States or if the VCP mutation originated in the USA and then radiated.

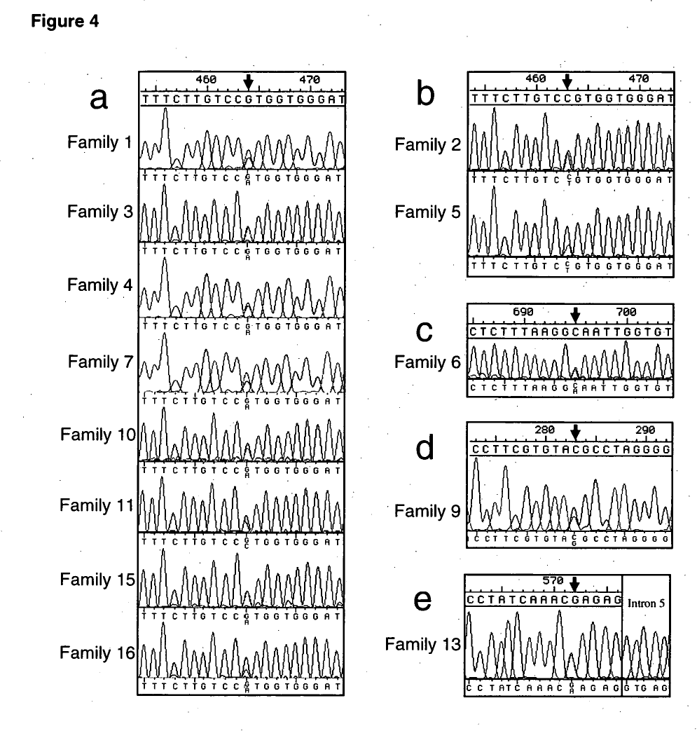

Using a candidate gene approach, GNE (UDP-N-acetylglucosamine 2-epimerase/N-acetylmannosamine kinase), which causes IBM2 or Nonaka myopathy and sialuria,^{3,4} and then several additional genes⁵ were excluded. Subsequently, six missense mutations (FIG. 4 and FIG. 5) within VCP (NM_{—}007126) were identified. VCP is also called CDC48 or p97 (a member of the AAA-ATPase super family—ATPase Associated with a variety of cellular Activities)⁶. Families 1, 3, 4, 7, 10, 15 and 16 share a 464 G>A (R155H) change in exon 5, whereas family 11 also has an alteration at base 464 but involving a G>C (R155P) change. Families 2 and 5 have an alteration at the first base of the same codon 463 C>T (R155C). Family 6 has a transition mutation 695 C>A (A232E) in exon 6. Family 9 has a base change in exon 3 at 283 C>G (R95G), whereas family 13 features a change at base 572 that is G>C (R191Q) in exon 5. The group A haplotype shares the 464 G>A (R155H) mutation and Group B share the 463 C>T (R155C) mutation. In families 4, 10 and 15 (who have the same VCP mutation as Group A, but unique haplotypes), their mutations probably arose independently from Group A. Families 6, 9, 11 and 13 do not share haplotypes and their VCP mutations are unique, therefore arising independently. Nevertheless, 10 of the 13 IBMPFD families have an amino acid change at codon 155 in VCP, which seems therefore, to be a mutation “hot spot”. Hence, identification of 6 distinctive cosegregating missense mutations within the gene encoding VCP identifies this as the genetic basis for IBMPFD.

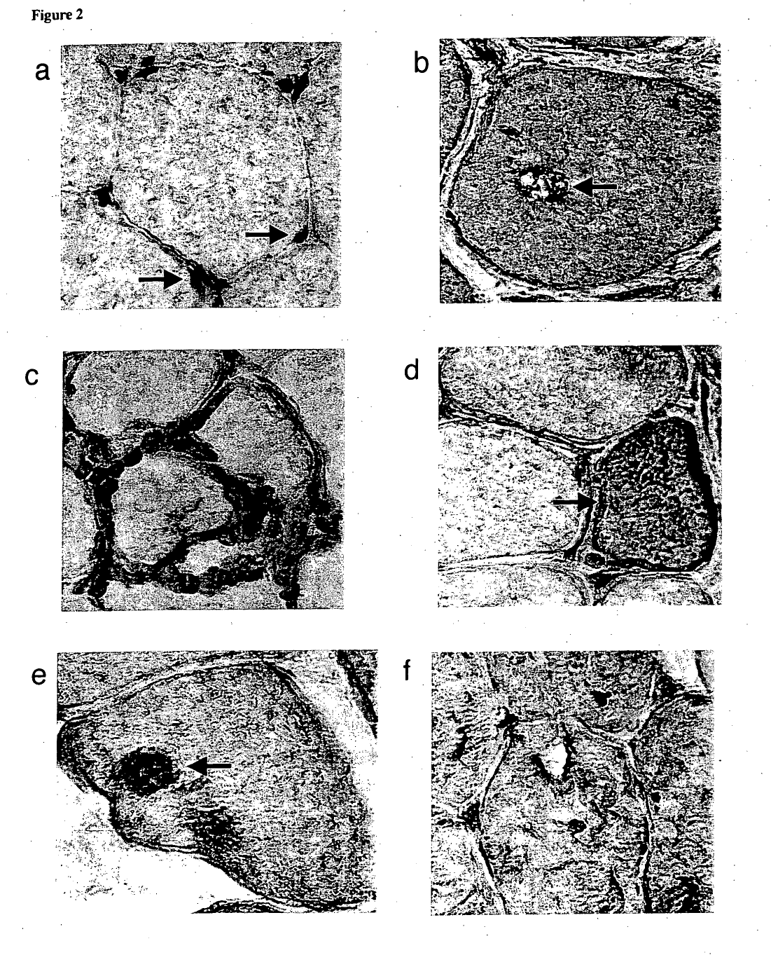

Immunohistochemistry of normal muscle sections with a polyclonal anti-VCP antibody showed staining of endomysial vessels, lipofuscin accumulations and to a mild degree, muscle fiber cytoplasm within muscle fibers (FIG. 2a). However, in sections of sporadic inclusion body myositis (s-IBM) muscle showed VCP staining localized to debris within inclusions and vacuoles (FIG. 2b). Interestingly, there was significant staining of VCP within inflammatory cells at the focal invasion sites of muscle fibers (FIG. 2c). VCP is up regulated in regenerating muscle fibers of s-IBM (FIG. 2d). In IBMPFD, VCP was localized in large or small rounded aggregates in scattered muscle fibers (FIG. 2e, f), including those with no clear vacuoles or other morphological changes. Thus, VCP is commonly present in aggregates from IBMPFD and s-IBM muscle, although the predominant pattern of localization for VCP differs between IBMPFD and s-IBM.

VCP mutations in families 1-7, 9-11, 13, 15, and 16 cluster in the N-terminal CDC48 domain (FIG. 3a), which is involved in ubiquitin binding^{7,8}. This highly structured N-domain forms two distinct regions⁹. VCP is highly conserved among species and the amino acid residues mutated in IBMPFD are conserved in the higher mammals (FIG. 3b). VCP forms a homohexamer where the D1/D2 domains bind in a head-to-tail ring⁹ allowing the N-terminal domain to undergo conformational changes without affecting the stability of the homohexamer ring structure. VCP missense mutations causing IBMPFD disrupt the double ψ barrel (R95G: family 9), the four-stranded β barrel (R155C/H/P: families 1-5,7,10,11,15), or the flexible linker (R191Q: family 13). Hence, the affected ubiquitin-binding domain may impair N-terminal domain binding of specific partner proteins. The family 6 mutation (A232E), within the α5 helix of the a/b sub-domain of the first AAA-ATPase domain (D1), is potentially more deleterious because the D1 domain provides the protein's main catalytic activity essential for hexamer formation¹⁰. In fact, affected individuals in family 6 have fractures and PDB at an earlier age and the myopathy seems especially aggressive.

VCP has been associated with several distinct and crucial cell protein pathways¹¹; namely cell cycle, homotypic membrane fusion, nuclear envelope reconstruction, postmitotic Golgi reassembly, DNA damage response, suppressor of apoptosis, and ubiquitin-dependent protein degradation¹²⁻¹⁸. VCP also binds to expanded poly-glutamine (poly-Q) protein aggregates^{18,19}. The poly-Q binding domain of human VCP maps to amino acid residues 142-200, which encompasses a region of the N-domain and linker (N domain to D1) that contains two of the mutations we identified¹⁸. A Drosophila VCP (ter94) loss-of-function mutant has been identified as a dominant suppressor of expanded poly-Q induced neuronal degeneration²⁰. The suppressive effects of the loss-of-function mutant did not appear to result from inhibition of poly-Q aggregate formation, but from the degree of VCP loss-of-function. This suggests that a gene dosage response for VCP expression is crucial to its function in expanded poly-glutamine (poly-Q) induced neuronal degeneration. To further support this, in transgenic Drosophila, where VCP levels were elevated, severe apoptotic cell death was induced, whereas homozygous VCP loss-of-function mutants were embryonic lethal²⁰.

VCP is an essential gene important for the cell cycle and apoptosis pathways, neither of which appear to be disrupted in IBMPFD, since affected individuals are obviously viable. Clues concerning the nature of the mutations we identified in VCP can be drawn from pathways that have been implicated in other aggresome-associated degenerative disorders, which all involve protein quality control and the ubiquitin protein degradation pathways²¹⁻²⁴. There are a number of independent studies supporting the fact that disruption of a specific function of VCP leads to inclusion body formation: 1) Experiments identifying the involvement of VCP in ERAD have shown that dysfunction of VCP causes vacuole and inclusion body formation, ultimately leading to cell death^{18,19,25}. 2) VCP has been found to interact directly with polyubiquitinated proteins¹⁸⁻²⁰. 3) VCP has been identified as co-localizing with ubiquitin-containing nuclear inclusions in the cerebral cortex from a number of neuronal degenerative disorders involving protein quality control and the ubiquitin protein degradation pathways, such as Huntington, Alzheimer, Creutzfeldt-Jakob, and Parkinson disease (in particular the Lewy bodies) as well as motor neuron disease with dementia²⁶. 4) Interestingly, mutations clustering in the ubiquitin-binding domain of sequestosome 1 (SQSTM1, p62)^{27,28} cause autosomal dominant Paget disease of the bone (PDB3). It is proposed that mutations in VCP, like SQSTM1, cause PDB by compromising ubiquitin-binding and are targeting similar cellular pathways or proteins. Furthermore, p62 has been shown to co-localize with inclusion bodies in a number of degenerative disorders^{22,23}. Thus, it seems likely that IBMPFD is a new member of the aggresome-associated disorders, and that mutations in VCP have identified a new link in the pathway that leads to aggresome formation. Since IBMPFD is a dominant progressive syndrome it is likely that the mutations we have identified are relatively subtle and that aging, oxidative stress and ER stress define a threshold whereby the IBMPFD phenotype is subsequently manifest. Rather than the mutations disrupting a normal function of VCP, they could add new toxic gain-of-functions that results in new VCP actions. Alternatively, the mutation could be a dominant negative that disrupts normal hexamer formation of the VCP protein.

In thirteen IBMPFD families only four amino acid residues (three in the N-terminal domain and one in the D1 domain) are mutated in VCP, suggesting either a mutation hot-spot in the N-domain or that VCP has such tight operational constraints that other types of mutation elsewhere are lethal. Indeed, homozygous loss-of-function mutants in Drosophila were embryonic lethal²⁰ and could explain the lack of a knockout mouse model for VCP. Our current findings identify VCP as having a new and crucial role in aggresome disease, particularly in the seemingly unrelated tissues affected in IBMPFD, and indicate that VCP has more specialized functions remaining to be characterized.

### Example 2

Abnormal aggregation of ubiquitin and valosin-containing protein in inclusion body myopathy associated with Paget disease of the bone and frontotemporal dementia.

Materials and Methods

Muscle MRI

Whole body muscle MRI was performed on a 1.5-tesla scanner, body coil (Philips Gyroscan Intera, Best, The Netherlands). The protocol included T1 TSE-sequence images (FOV 530 mm, slice thickness 5 mm, matrix 217×512, TR 450 ms, TE 17 ms) and transverse T2 TSE SPIR-sequence images (FOV 530 mm, slice thickness 5 mm, matrix 205×256, TR 2600 ms, TE 80 ms) for the upper extremities and the upper chest transverse; transverse T1 TSE-sequence images (FOV 400 mm, slice thickness 5 mm, matrix 217×512, TR 450 ms, TE 17 ms) and transverse T2 TSE SPIR-sequence images (FOV 400 mm, slice thickness 5 mm, matrix 205×256, TR 2600 ms, TE 80 ms) for the abdomen and pelvis; transverse T1 TSE-sequence images (FOV 400 mm, slice thickness 5 mm, matrix 217×512, TR 450 ms, TE 17 ms) and transverse T2 TSE SPIR-sequence images (FOV 400 mm, slice thickness 5 mm, matrix 205×256, TR 2600 ms, TE 80 ms) for the thighs and lower legs.

VCP Mutation Analysis

Informed consent in writing was obtained from our patient. Isolation of DNA as well as VCP mutation analysis was performed as decribed previously³⁰.

Muscle Biopsy, Antibodies, Immunohistochemistry and Immunoblotting

An open diagnostic biopsy was taken from the left vastus lateralis muscle. Six μm thick cryostat sections of snap-frozen unfixed muscle were stained by standard procedures. Indirect immunofluoresecence analysis and immunoblotting of skeletal muscle were performed as described previously^{32,33}. The following primary antibodies were used: VCP rabbit antiserum (kind gift of Dr. Chou-Chi Li) and anti-ubquitin antibody clone FPM1 (Novocastra), a monoclonal mouse antibody raised against ubiquitin. All specimens were examined and digital pictures were acquired using a Nikon E800 microscope (Nikon, Düsseldorf, Germany) equipped with a CCD camera.

Results

Clinical Phenotype

A 54-year-old female presented with a 30 year history of progressive muscle weakness and atrophy predominantly affecting her shoulder girdle, trunk and distal leg muscles. She is the first off-spring of a consangineous German family in which her parents are first degree cousins. She has three healthy younger siblings and a clinically not affected 32 year old daughter. Her further family history was informative and negative. Paget disease of the bone confined to the first lumbar vertebra was histologically diagnosed in 2002. She was not demented and no overt behavioral abnormalities. However, a detailed neuropsychological evaluation in February 2004 revealed a performance far below average (>2 standard deviations below mean) in the labyrinth task testing for anticipation, and below average (>1 standard deviation below mean) in the figural memory and naming tests, suggesting mild fronto-temporal cognitive dysfunction. Brain MRI gave normal results. Neurological examination showed severe weakness and atrophy of her scapular fixator muscles (deltoid, rhomboid, supra and infraspinatus) and trunk extensors. In addition, she had slight to moderate muscle weakness of her finger extensor, hip flexor and distal leg muscles. Seum CPK was within normal limits. Whole body MRI demonstrated widespread muscular involvement with pronounced signal changes in her erector spinae, hamstring and calf muscles.

The R155H Mutation is Associated With to VCP- and Ubiquitin-Positive Protein Aggregation

DNA mutation analysis revealed a heterozygous 464 G→A nucleotide substitution in exon 5 of the VCP gene that causes an amino acid substitution from arginine to histidine (R155H). The missense mutation resides in the N-terminal part of the gene that encodes the CDC48 domain, which is involved in ubiquitin binding (data not shown)⁷. Morphological analysis of the muscle biopsy showed myopathic changes consisting of type I fiber predominance, atrophic and hypertrophic fibers, slight broadening of connective tissue, de- and regenerating fibers and a single ragged red fiber (data not shown). Furthermore, there were several fibers with rimmed vacuoles but no inflammatory infiltrates.

VCP immunostaining of normal human skeletal muscle showed a weak cytoplasmic staining of muscle fibers as well as a moderate labelling of endomysial capillaris (data not shown). Double immunofluorescence analysis of skeletal muscle from our patient revealed a subset of fibers (<5%) containing single or multiple cytoplasmic foci of VCP- and ubiquitin-positive protein aggregate. To address the issue whether the increased VCP immunostaining is paralleled by an altered VCP protein expression, Western blotting of total protein extracts from normal and IBMPFD muscle was performed. Immunoblotting using a VCP antibody detected a single band corresponding in size to 97 kDa in both normal and diseased muscle. Interestingly, the signal intensity in IBMPFD muscle was more intense than in normal muscle indicating a marked increase of VCP protein level.

As described throughout this application, mutations of the VCP gene on chromosome 9p21.1-p12 cause autosomal dominant IBMPFD. In 13 North American families with IBMPFD, six VCP missense mutations have been identified, which are exclusively found in symptomatic individuals.

A 54-year-old German IBMPFD patient with special focus on skeletal muscle pathology was studied. Mutation analysis revealed a heterozygous 464 G→A nucleotide substitution (R155H) in exon 5 of the VCP gene. The negative family history suggests that the G→A nucleotide substitution is due to a de novo mutation in the reported patient. In line with previous data showing that 10 of 13 affected families have an amino acid change at codon 155 (R155H, n=7; R155P, n=1; R155C, n=2) the identification of the “common” R155H mutation in our sporadic case further supports that codon 155 is a mutation hot spot in VCP³⁰.

Clinical and MRI analysis documented a widespread muscular patholoy with predominant involvement of the shoulder girdle, erector spinae, hamstrings and calf muscles. Even though the diagnostic muscle biopsy was taken from a clinically not affected muscle, the morphological analysis revealed myopathic changes along with fibers containing rimmed vacuoles. Indirect immunofluorescence detected a subset of muscle fibers with VCP-positive, cytoplasmic protein aggregates. Furthermore, Western blot analysis demonstrated that aberrant VCP accumulation pattern is associated with an increase in VCP protein level in IBMPFD muscle. Since the R155H mutation resides in the N-terminal part of the VCP gene that encodes the CDC48 domain, which is involved in ubiquitin binding⁷, double staining with antibodies raised against VCP and ubiquitin was performed. The vast majority of VCP-positive protein aggregates also showed a strong ubiquitin labelling. However, no VCP- and ubiquitin-positive intranuclear inclusions, which have been described in a number of neuronal degenerative disorders, could be detected in skeletal muscle tissue from our patient.

Taken together, the findings indicate that the expression of the “common” R155H VCP mutation interferes with the ubiquitin-proteasome degradation pathway thereby leading to abnormal VCP protein aggregation in human skeletal muscle.

The references cited throughout the specification are herewith incorporated by reference in their entirety.

### REFERENCES

1. Kimonis, V. E. et al. Clinical and molecular studies in a unique family with autosomal dominant limb-girdle muscular dystrophy and Paget disease of bone. Genet. Med. 2, 232-241 (2000).
2. Kovach, M. J. et al. Clinical delineation and localization to chromosome 9p13.3-p12 of a unique dominant disorder in four families: hereditary inclusion body myopathy, Paget disease of bone, and frontotemporal dementia. Mol. Genet. Metab. 74, 458-475 (2001).
3. Eisenberg, I. et al. The UDP-N-acetylglucosamine 2-epimerase/N-acetylmannosamine kinase gene is mutated in recessive hereditary inclusion body myopathy. Nat. Genet. 29, 83-87 (2001).
4. Seppala, R., Lehto, V. P. & Gahl, W. A. Mutations in the human UDP-N-acetylglucosamine 2-epimerase gene define the disease sialuria and the allosteric site of the enzyme. Am. J. Hum. Genet. 64, 1563-1569 (1999).
5. Watts, G. D. J., Thorne, M., Kovach, M. J., Pestronk, A. & Kimonis, V. E. Clinical and Genetic Heterogeneity in Chromosome 9p associated Hereditary Inclusion Body Myopathy: Exclusion of GNE and three other candidate genes. Neuromuscul. Disord. 13, 559-567 (2003).
6. Beyer, A. Sequence analysis of the AAA protein family. Protein Sci. 6, 2043-2058 (1997).
7. Dai, R. M. & Li, C. C. Valosin-containing protein is a multi-ubiquitin chain-targeting factor required in ubiquitin-proteasome degradation. Nat. Cell Biol. 3, 740-744 (2001).
8. Rape, M. et al. Mobilization of processed, membrane-tethered SPT23 transcription factor by CDC48(UFD1//NPL4), a ubiquitin-selective chaperone. Cell 107, 667-677 (2001).
9. Zhang, X. et al. Structure of the AAA ATPase p97. Mol. Cell 6, 1473-1484 (2000).
10. Wang, Q., Song, C. & Li, C. C. Hexamerization of p97-VCP is promoted by ATP binding to the D1 domain and required for ATPase and biological activities. Biochem. Biophys. Res. Commun. 300, 253-260 (2003).
11. Wang, Q., Song, C. & Li, C. C. Molecular perspectives on p97-VCP: progress in understanding its structure and diverse biological function. Journal of Structural Biology In Press, Corrected Proof (2003) (http://www.sciencedirect.com/science/article/B6WM5-4B8B9KY-2/2/28f53b7451987b8a6390ccd97f305a19).
12. Hetzer, M. et al. Distinct AAA-ATPase p97 complexes function in discrete steps of nuclear assembly. Nat. Cell Biol. 3, 1086-1091 (2001).
13. Rabinovich, E., Kerem, A., Frohlich, K. U., Diamant, N. & Bar-Nun, S. AAA-ATPase p97/Cdc48p, a cytosolic chaperone required for endoplasmic reticulum-associated protein degradation. Mol. Cell. Biol. 22, 626-634 (2002).
14. Rabouille, C. et al. Syntaxin 5 is a common component of the NSF- and p97-mediated reassembly pathways of Golgi cisternae from mitotic Golgi fragments in vitro. Cell 92, 603-610 (1998).
15. Kondo, H. et al. p47 is a cofactor for p97-mediated membrane. Nature 388, 75-78 (1997).
16. Meyer, H. H., Shorter, J. G., Seemann, J., Pappin, D. & Warren, G. A complex of mammalian ufd1 and np14 links the ubiquitin and nuclear transport pathways. EMBO J. 19, 2181-2192 (2000).
17. Jarosch, E., Geiss-Friedlander, R., Meusser, B., Walter, J. & Sommer, T. Protein dislocation from the endoplasmic reticulum—pulling out the suspect. Traffic 3, 530-536 (2002).
18. Hirabayashi, M. et al. VCP/p97 in abnormal protein aggregates, cytoplasmic vacuoles, and cell death, phenotypes relevant to neurodegeneration. Cell Death Differ. 8, 977-984 (2001).
19. Kobayashi, T., Tanaka, K., Inoue, K. & Kakizuka, A. Functional ATPase activity of p97/valosin-containing protein (VCP) is required for the quality control of endoplasmic reticulum in neuronally differentiated mammalian PC12 cells. J. Biol. Chem. 277, 47358-47365 (2002).
20. Higashiyama, H. et al. Identification of ter94, Drosophila VCP, as a modulator of polyglutamine-induced neurodegeneration. Cell Death Differ. 9, 264-273 (2002).
21. Layfield, R., Alban, A., Mayer, R. J. & Lowe, J. The ubiquitin protein catabolic disorders. Neuropathol. Appl. Neurobiol. 27, 171-179 (2001).
22. Garcia-Mata, R., Gao, Y. S. & Sztul, E. Hassles with taking out the garbage: aggravating aggresomes. Traffic 3, 388-396 (2002).
23. Donaldson, K. M. et al. Ubiquitin-mediated sequestration of normal cellular proteins into polyglutamine aggregates P62 and the sequestosome, a novel mechanism for protein metabolism. Proc. Natl. Acad. Sci. U.S.A. 100, 8892-8897 (2003).
24. Wood, J. D., Beaujeux, T. P. & Shaw, P. J. Protein aggregation in motor neurone disorders. Neuropathol. Appl. Neurobiol. 29, 529-545 (2003).
25. Nagahama, M. et al. SVIP is a novel VCP/p97-interacting protein whose expression causes cell vacuolation. Mol. Biol. Cell. 14, 262-273 (2003).
26. Mizuno, Y., Hori, S., Kakizuka, A. & Okamoto, K. Vacuole-creating protein in neurodegenerative diseases in humans. Neurosci. Lett. 343, 77-80 (2003).
27. Hocking, L. J. et al. Domain-specific mutations in sequestosome 1 (SQSTM1) cause familial and sporadic Paget's disease. Hum. Mol. Genet. 11, 2735-2739 (2002).
28. Laurin, N., Brown, J. P., Morissette, J. & Raymond, V. Recurrent mutation of the gene encoding sequestosome 1 (SQSTM1/p62) in Paget disease of bone. Am. J. Hum. Genet. 70, 1582-1588 (2002).
29. Yabe, H., Singer, F. R., Tucker, W. S., Jr. & Mills, B. G. Paget-like inclusions in osteopetrosis and hereditary neuromuscular and skeletal disease. Eight Annual Meeting of the American Soceity of Bone and Mineral Research, A221 (1986).
30. Watts G D, Wymer J, Kovach M J et al. Inclusion body myopathy associated with Paget disease of bone and frontotemporal dementia is caused by mutant valosin-containing protein. Nat Genet 2004;36:377-381.
31. Waggoner B, Kovach M J, Winkelman M et al. Heterogeneity in familial dominant Paget disease of bone and muscular dystrophy. Am J Med Genet 2002;108:187-191.
32. Fischer D, Schroers A, Blumcke I et al. Consequences of a novel caveolin-3 mutation in a large German family. Ann Neurol 2003;53:233-241.
33. Schröder R, Goudeau B, Simon M C et al. On noxious desmin: functional effects of a novel heterozygous desmin insertion mutation on the extrasarcomeric desmin cytoskeleton and mitochondria. Hum Mol Genet 2003;12:657-669.

We claim:

1. A method of diagnosing the presence or absence of inclusion body myopathy-Paget bone disease-frontotemporal dementia syndrome (IBMPFD) comprising the steps of obtaining a biological sample from an individual, and analyzing a nucleic acid encoding vasolin containing protein (VCP) in the biological sample for alterations, wherein an alteration in the nucleic acid encoding VCP compared to a control nucleic acid sample obtained from an individual not affected with the IBMPFD is indicative of the presence of IBMPFD.

2. The method of claim 1, wherein the alteration in the nucleic acid encoding VCP comprises an alteration in Exon 3, Exon 5, or Exon 6 of a VCP gene.

3. The method of claim 1, wherein the nucleic acid encoding VCP comprises SEQ ID NO: 2.

4. The method of claim 3, wherein the alteration is a mutation selected from the group consisting of 464 G>A; 464 G>C; 463 C>T; 695 C>A; 283 C>G; and 572 G>C.

5. The method of claim 1, wherein the alteration results in an amino acid change in VCP (SEQ ID NO: 1) selected from the group consisting of R155H, R155P, R155C, A232E, R95G, and R191Q.

6. A method for detecting the presence or absence of an alteration in a gene encoding vasolin containing protein (VCP) in a biological sample, comprising the steps of:
a. analyzing a biological test sample containing a gene encoding VCP;
b. comparing the results of the analysis of the biological sample with the results of analysis of a control sample, wherein the control sample comprises a gene encoding VCP without an alteration; and
c. determining the presence or absence of an alteration in the test sample compared to the absence of the alteration in the control sample.

7. A method for diagnosing the presence or absence of inclusion body myopathy-Paget bone disease-frontotemporal dementia syndrome (IBMPFD) in a patient comprising the steps of:
a. contacting a biological test sample obtained the patient with a nucleic acid probe, wherein the nucleic acid probe detects at least one alteration in a gene encoding VCP;
b. maintaining the biological test sample and the nucleic acid probe under conditions suitable for hybridization;
c. detecting hybridization between the biological test sample and the nucleic acid probe;
d. and comparing hybridization in the biological test sample from the patient to a control sample,
wherein the presence of hybridization between the biological test sample and the nucleic acid probe compared to the control sample is indicative of the presence of IBMPDF in the patient.

8. The method of claim 7, wherein the nucleic acid probe is labeled.

9. The method of claim 8, wherein the label comprises a fluorescent, radioactive, or enzymatic label.

10. A method for diagnosing the presence or absence of inclusion body myopathy-Paget bone disease-frontotemporal dementia syndrome (IBMPFD) in a patient comprising the steps of:
a. performing a nucleic acid amplification of a biological test sample with oligonucleotide primers capable of amplifying a gene encoding VCP;
b. analyzing the amplified nucleic acid fragments of the gene encoding VCP; and
c. comparing the amplified nucleic acid fragments detected in step b) with amplified nucleic acid fragments of a control sample,
wherein the presence of an alteration in the biological test sample compared to the control sample is indicative of the presence of IBMPDF in the patient.

11. The method of claim 10, comprising the additional step of sequencing the amplified nucleic acid fragments.

12. The method of claim 6, 7, or 10, wherein the alteration in the nucleic acid encoding VCP comprises an alteration in Exon 3, Exon 5, or Exon 6 of a VCP gene.

13. The method of claim 6, 7, or 10, wherein the nucleic acid encoding VCP comprises SEQ ID NO: 2.

14. The method of claim 6, 7, or 10, wherein the alteration is a mutation selected from the group consisting of 464 G>A; 464 G>C; 463 C>T; 695 C>A; 283 C>G; and 572 G>C.

15. The method of claim 6, 7, or 10, wherein the alteration results in an amino acid change in VCP (SEQ ID NO: 1) selected from the group consisting of R155H, R155P, R155C, A232E, R95G, and R191Q.

16. An isolated VCP nucleic acid encoding a mutant VCP having an amino acid change selected from the group consisting of R155H, R155P, R155C, A232E, R95G, and R191Q.

17. The method of claim 1, 6, 7, or 10, wherein the biological sample is a bodily fluid sample selected from the group consisting of blood, saliva, semen, vaginal secretion, cerebrospinal fluid and amniotic bodily fluid sample.

18. The method of claim 1, 6, 7, or 10, wherein the biological sample is a tissue sample selected from the group consisting of epithelial, muscular, neuronal, bone, chorionic villous, or connective tissue sample.

19. A method of diagnosing inclusion body myopathy comprising immunohistochemical staining of muscle sections with anti-VCP antibody, wherein localization of VCP within inclusion bodies is indicative of inclusion body myopathy.

20. The method of claim 17, wherein the inclusion body myopathy is associated with IBMPFD.

21. The method of claim 18, further comprising immunohistochemical staining with anti-ubiquitin antibody.

22. A kit comprising one or more reagents for detecting an alteration in a gene encoding VCP.

23. The kit of claim 22, comprising nucleic acid probes or primers capable of detecting an alteration in a gene encoding VCP, and wherein the alteration results in an amino acid change in VCP (SEQ ID NO: 1) selected from the group consisting of R155H, R155P, R155C, A232E, R95G, and R191Q.

24. A method of diagnosing the presence or absence of inclusion body myopathy-Paget bone disease-frontotemporal dementia syndrome (IBMPFD) in an individual, wherein IBMPFD is associated with one or more alterations in the gene encoding vasolin containing protein (VCP), comprising analyzing a biological sample obtained from the individual for alterations in a gene encoding VCP, wherein the presence of an alteration in the gene is indicative of the presence of IBMPFD, and the absence of an alteration in the gene is indicative of the absence of IBMPFD.

25. The method of claim 24 wherein the alteration results in an amino acid change in VCP (SEQ ID NO: 1) selected from the group consisting of R155H, R155P, R155C, A232E, R95G, and R191Q.
